(19) United States
(12) Patent Application Publication
(10) Pub. No.: US 2007/0208400 A1
(43) Pub. Date: Sep. 6, 2007

(54) SYSTEM AND METHOD FOR PROVIDING CELL SPECIFIC LASER THERAPY OF ATHEROSCLEROTIC PLAQUES BY TARGETING LIGHT ABSORBERS IN MACROPHAGES

(75) Inventors: Seemantini K. Nadkarni, Boston, MA (US); Guillermo J. Tearney, Cambridge, MA (US); Brett Eugene Bouma, Quincy, MA (US); Benjamin J. Vakoc, Cambridge, MA (US); Richard Rox Anderson, Boston, MA (US)

Correspondence Address: DORSEY & WHITNEY LLP;INTELLECTUAL PROPERTY DEPARTMENT, 250 PARK AVENUE, NEW YORK, NY, 10177, US

(73) Assignee: The General Hospital Corporation, Boston, MA (US)

(21) Appl. No.: 11/680,962

(22) Filed: Mar. 1, 2007

Related U.S. Application Data
(60) Provisional application No. 60/778,336, filed on Mar. 1, 2006.
(60) Provisional application No. 60/783,599, filed on Mar. 17, 2006.

(51) Int. Cl. A61F 2/00; A61N 5/06

(52) U.S. Cl. 607/100; 977/915; 607/89

(57) ABSTRACT

An apparatus and method according to the present invention can be provided, e.g., for a cell specific laser therapy of atherosclerotic plaques, particularly to systems and methods for targeting endogenous light absorbers present within plaque macrophages and exogenous nanoparticle targeting. In one exemplary embodiment, an electro-magnetic radiation can be forwarded to an anatomical structure. The electromagnetic radiation may have at least one property configured to (a) modify at least one characteristic of at least one first cell, and (b) minimize any modification of and/or modify at least one characteristic of at least second cell. The first and second cells may be different from one another, the characteristics of the first and second cells can be different from one another, and the first cell and/or the second cell may have at least one macrophage feature, and the characteristic of the at least one first cell and/or the at least one second cell can be temperature. According to still another exemplary embodiment, a location associated with the first cell and the second cell can be determined. For example, the electromagnetic radiation can be forwarded in a vicinity of the location.

### CROSS-REFERENCE TO RELATED APPLICATION(S)

This application is based upon and claims the benefit of priority from U.S. Patent Application Ser. No. 60/778,336 filed Mar. 1, 2006 and U.S. Patent Application Ser. No. 60/783,599, filed Mar. 17, 2006, the entire disclosures of which are incorporated herein by reference.

### FIELD OF THE INVENTION

The present invention relates to systems and methods for providing a cell specific laser therapy of atherosclerotic plaques, particularly to systems and methods for targeting endogenous light absorbers present within plaque macrophages and exogenous nanoparticle targeting. In addition, the present invention further relates to systems and methods for biodistribution of noble-metal nanoparticles and evaluation of an optical signature associated with the nanoparticle distribution in macrophage-rich tissues.

### BACKGROUND OF THE INVENTION

### Selective Cell Targeting for Therapy

An ability to target specific cells to induce selective localized cell necrosis while maintaining the health and integrity of the surrounding tissue may have significant therapeutic benefits for a variety of diseases. The early detection and selective targeting of cancerous cells can potentially limit tumor growth and proliferation resulting in better clinical outcomes. (See A. F. Chambers et al, “Critical steps in hematogenous metastasis: an overview,” Surg Oncol Clin N Am. 2001;10, pp. 243-55, vii). Considerable effort has been made in identifying factors involved in tumor metastasis, and the identification and selective killing of extravasated cancerous cells may potentially control tumor metastasis. (See A. F. Chambers, “The metastatic process: basic research and clinical implications,” Oncol Res. 1999; 11, pp. 161-8). Selective cell targeting also has potential therapeutic applications in ischemic cardiovascular, cerebrovascular and peripheral artery disease. The selective killing of macrophages, which are implicated in atherogenesis and plaque rupture (see I. Tabas, “Consequences and therapeutic implications of macrophage apoptosis in atherosclerosis: the importance of lesion stage and phagocytic efficiency,” Arterioscler Thromb Vasc Biol. 2005;25, pp. 2255-64), may have far reaching therapeutic benefits for plaque stabilization, consequently reducing the risk of ischemic events. In inflammatory diseases such as rheumatoid arthritis which affects the bones, blood vessels, skin, heart and lungs, localized therapy by selectively killing infiltrated macrophages and inflammatory cells may reduce progression of this debilitating chronic disease.

A number of ocular diseases, resulting in degeneration of the retina, are characterized by the loss of visual receptors—rods and cones, accompanied by focal proliferation of retinal pigment epithelium (RPE) cells. By selectively damaging pigmented RPE cells while maintaining the viability of surrounding cells, significant treatment benefits may be obtained. (See R. Brinkmann et al., “Origin of retinal pigment epithelium cell damage by pulsed laser irradiance in the nanosecond to microsecond time regimen,” Lasers Surg Med. 2000;27, pp. 451-64). The selective targeting of cells has several applications in dermatology for the treatment of microvessels in hemangiomas, removal of hair follicles, selective destruction of sebaceous glands for acne treatment, and selective heating of adipocytes for subcutaneous fat removal. (See D. Manstein et al., “Selective photothermolysis of lipid rich tissue. Annual meeting of Lasers in Surgery and Medicine,” 2001; Supplement 13:Abs no. 17; and R. R. Anderson et al., “Selective photothermolysis of cutaneous pigmentation by Q-switched Nd: YAG laser pulses at 1064, 532, and 355 nm,” J Invest Dermatol. 1989;93, pp. 28-32).

### Exemplary Techniques for Selective Cell Targeting

Recently, several research groups have undertaken significant research efforts to develop methods for delivering localized therapy to specific cells implicated in disease progression. Exemplary approaches to achieve selective cell damage include the use of light-activated drugs, nanomaterials and laser-based techniques for cell specific therapy. Photodynamic therapy (PDT) can use immunoconjugated photosensitizers that become toxic to cells only in areas exposed to light. (See M. Del Governatore et al., “Experimental photoimmunotherapy of hepatic metastases of colorectal cancer with a 17.1A chlorin(e6) immunoconjugate,” Cancer Res. 2000;60, pp. 4200-5). In particular, the photosensitiser, Motexafin lutetium which is taken up by atherosclerotic plaques, can cause macrophage and smooth muscle cell apoptosis upon light activation in animals.(M. Hayase et al., “Photoangioplasty with local motexafin lutetium delivery reduces macrophages in a rabbit post-balloon injury model,” Cardiovasc Res. 2001;49, pp. 449-55).

With the advent of nanotechnology, certain materials such as noble metal and magnetofluorescent nanoparticles, and quantum dots can be used enabling in vivo identification of cells as well as for therapeutic use. (See G. M. Whitesides. “The ‘right’ size in nanobiotechnology,” Nat Biotechnol. 2003;21, pp. 1161-5). Nanomaterials with precise biological functions are being developed for disease diagnosis and for delivering pharmacologic agents for localized cell therapy. (See R. Weissleder et al., “Cell-specific targeting of nanoparticles by multivalent attachment of small molecules,” Nat Biotechnol. 2005;23:1418-23; and N. Tsapis, “Trojan particles: large porous carriers of nanoparticles for drug delivery,” Proc Natl Acad Sci USA. 2002;99, pp. 12001-5).

The use of laser light for selective cell therapy has been investigated in a variety of applications utilizing exogenous and endogenous light absorbers. Microparticles and nanoparticles which are engulfed by target cells have been utilized to achieve highly localized cell damage by delivering nanosecond laser pulses. (See C. M. Pitsillides et al., “Selective cell targeting with light-absorbing microparticles and nanoparticles,” Biophys J. 2003;84, pp. 4023-32). The use of endogenous chromophores in cells as near infrared light absorbers has been investigated in ophthalmology to selectively damage pigmented cells of the RPE, and in dermatology selective targeting of melanocytes with short laser pulses has been demonstrated for treating pigmented skin lesions. (See R. R. Anderson et al., “Selective photothermolysis of cutaneous pigmentation by Q-switched Nd: YAG laser pulses at 1064, 532, and 355 nm,” J Invest Dermatol. 1989;93, pp. 28-32).

### Selective Cell Targeting in Vulnerable Atherosclerotic Plaque

Atherosclerosis is a systemic disease and the rupture of atherosclerotic plaque is a major mechanistic precursor to acute coronary syndromes, ischemic stroke and peripheral artery disease. Macrophages are implicated in every stage of atherosclerosis from lesion initiation to clinical presentation. (See R. Ross, “Atherosclerosis—an inflammatory disease,” N Engl J Med. 1999;340, pp. 115-26; and P. Libby, “Inflammation in atherosclerosis,” Nature, 2002;420, pp. 868-74). In early lesions, macrophages ingest lipid causing the accumulation of lipid droplets in the cytoplasm, resulting in the formation of arterial foam cells. Apoptosis of macrophages can produce the thrombogenic necrotic core in advanced unstable lesions, as shown in FIG. 1.

The most common plaque type associated with acute myocardial infarction and acute coronary events is the thin-capped fibroatheroma. This type of plaque can contains a fibrous cap 100 overlying a lipid rich necrotic core 105. Macrophages 110 and 115 can be the cells responsible for plaque instability in these lesions, as they secrete matrix metalloproteinases that digest collagen, weaken the fibrous cap 100, thereby increasing the propensity of necrotic core fibroatheroma rupture. (See P. Libby, “Inflammation in atherosclerosis,” Nature, 2002;420, pp. 868-74). Furthermore macrophages express tissue factor, a known procoagulant, and have been found to be preferentially located close to the luminal surface in culprit lesions of patients with acute myocardial infarction and acute coronary syndromes. (See B. D. MacNeill et al., “Focal and multi-focal plaque macrophage distributions in patients with acute and stable presentations of coronary artery disease,” J Am Coll Cardiol. 2004;44, pp. 972-9).

Since macrophages generally can play a crucial role in plaque rupture and thrombosis, the reduction in numbers of these cells potentially stabilizes the plaque. Systemic therapy using lipid-lowering statins can markedly reduce the occurrence of acute coronary events and stroke resulting from plaque rupture without causing a significant change in arterial stenosis. This treatment benefit may be achieved by a stabilization of the plaque resulting from the reduction in numbers of macrophages and the subsequent accumulation of collagen fibrils in the lipid pool. (See P. Libby et al., “Stabilization of atherosclerotic plaques: new mechanisms and clinical targets,” Nat Med. 2002;8, pp. 1257-62). The combination of systemic statin therapy with localized plaque therapy to induce macrophage death while maintaining the integrity of the epithelium may reduce the threat of atherosclerotic plaque rupture and subsequent acute coronary events in patients.

### Selective Cell Targeting via Administration of Metal Nanoparticles

Noble-metal nanoparticles can generally comprise one class of optical contrast agents that may enhance macrophage visibility in situ. The ability of gold and silver nanoparticles to enhance both linear and nonlinear optical processes at low average laser powers, as well as their high biocompatibility, indicates that these particles may be useful optical contrast agents in living patients. Nanoparticles can be used as contrast agents to enhance various imaging techniques. Their small diameter, 5-20 nanometers, allows diffusion through cellular junctions and capillaries. MRI contrast agents, notably ultrasmall (15-20 nm) superparamagnetic particles of iron oxide (USPIOs), have been shown to penetrate the endothelium and also be selectively phagocytosed by macrophages in atherosclerotic plaques. (See S. G. Ruehm et al. “Magnetic resonance imaging of atherosclerotic plaque with ultrasmall superparamagnetic particles of iron oxide in hyperlipidemic rabbits,” Circulation, 2001;103, pp. 415-22). Based on this exemplary data, atherosclerotic plaque macrophages may also selectively uptake noble-metal nanoparticles.

When resident in tissue, noble-metal nanoparticles may provide a high optical signal in confocal microscopy and optical coherence tomography, due to their high elastic scattering efficiency. In addition, nonlinear optical phenomena associated with resonantly excited noble-metal nanoparticles may be exploited for diagnosis. When the laser field frequency coincides with the plasmon frequency of a noble-metal nanoparticle, a large field enhancement can be achieved in a close proximity to the particle surface. This effect can be utilized to significantly increase the Raman scattering cross section, two-photon auto-fluorescence, and second- and third-harmonic generation of adsorbed molecules. These locally enhanced processes may provide unique optical signatures that provide information on both the nanoparticle distribution as well as regional chemical composition.

Accordingly, there is a need to overcome the deficiencies described herein above.

### OBJECTS AND SUMMARY OF THE INVENTION

To address and/or overcome the above-described problems and/or deficiencies as well as other deficiencies, systems and methods can be provided for facilitating a cell specific laser therapy of atherosclerotic plaques. For example, systems and methods can be provided for targeting endogenous light absorbers present within plaque macrophages and exogenous nanoparticle targeting. In addition, systems and methods may be provided for biodistribution of noble-metal nanoparticles and evaluation of an optical signature associated with the nanoparticle distribution in macrophage-rich tissues.

Such deficiencies can be addressed using the exemplary embodiments of the present invention. In one exemplary embodiment of the present invention, In one exemplary embodiment, an electromagnetic radiation can be forwarded to an anatomical structure. The electromagnetic radiation may have at least one property configured to (a) modify at least one characteristic of at least one first cell, and (b) minimize any modification of and/or modify at least one characteristic of at least second cell. The first and second cells may be different from one another, the characteristics of the first and second cells can be different from one another, and the first cell and/or the second cell may have at least one macrophage feature, and the characteristic of the at least one first cell and/or the at least one second cell can be temperature.

The property may include wavelength, average power, instantaneous power, pulse duration and/or total exposure duration. The wavelength may be approximately the same as a wavelength of an absoption characteristic of a compound within the first cell. The pulse duration may cause the characteristic to be be confined approximately within the first cell. The power can causes the characteristic to irreversably damage at least one portion of the first cell. The charactristic of the first cell may be greater than a temperature that causes a damage to at least one portion of the first cell. The damage to the portion of the first cell may be irreversible. The first cell may be situated within a vascular wall. The vascular wall can be a part of a coronary artery.

According to another exemplary embodiment of the present invention, the arrangement can be provided in a catheter. The macrophage feature may be a lysozome containing at least one lipid. The lipid can include at least one low density lipo protein (“LDL”), oxidized LDL, cholesterol or cholesterol ester. The macrophage feature may be a lysozome containing nacrotic debris, a multitude of lysozomes and/or at least one nanoparticle. The apparatus according to claim 1. The nanoparticle may be provided within the cell and/or a lysozome. A size of a single nanoparticle can be between 1 and 20 nanometer. The nanoparticle may be comprised of metal, nobel metal, ultra-small para-magnetic iron oxide, gold and/or silver. The nanoparticle can be administered to a subject interveneously. When the electromagnetic radiation is applied on the nanoparticle, a surface plasmon may be generated. According still another exemplary embodiment of the present invention, the electromagnetic radiation can be forwarded externally from a body of a subject. Further, the macrophage feature provided may be within a fiberous cap.

According to yet another exemplary embodiment, a location associated with the first cell and the second cell can be determined. For example, the electromagnetic radiation can be forwarded in a vicinity of the location. The location can be determined based on an image of at least one portion of the anatomical structure. An arrangement can be used to determine the location which may include a coherence ranging arrangement, a speckle analysis arrangement, a thermal imaging arrangement, and/or a spectroscopy arrangement.

These and other objects, features and advantages of the present invention will become apparent upon reading the following detailed description of embodiments of the invention, when taken in conjunction with the appended claims.

### BRIEF DESCRIPTION OF THE DRAWINGS

Further objects, features and advantages of the present invention will become apparent from the following detailed description taken in conjunction with the accompanying figures showing illustrative embodiments of the present invention, in which:

FIG. 1 are exemplary microscopic images of lipid filled macrophages in human atherosclerotic plaques;

FIG. 2 is a cross-sectional view of a non-specific heating of a tissue during laser ablation thereof;

FIG. 3 is a cross-sectional view of a cell for a cell specific heating;

FIG. 4 is a graph showing a selective optical absorption of subcutaneous fat measured using spectro-photometric measurements, with a differential absorption spectrum between fat and water;

FIG. 5A is a schematic diagram demonstrating a cell-specific therapy system according to one exemplary embodiment of the present invention;

FIG. 5B is a schematic diagram demonstrating the cell-specific therapy system according to another exemplary embodiment of the present invention;

FIG. 6 is a schematic diagram of the cell-specific laser therapy catheter according to one single fiber embodiment of the present invention;

FIG. 7 is a schematic diagram of the cell-specific laser therapy catheter according to another single fiber embodiment of the present invention that is in close proximity to the arterial wall;

FIG. 8 is a schematic diagram of the cell-specific laser therapy catheter according to another single fiber embodiment of the present invention that is encompassed within an exemplary balloon arrangement;

FIG. 9 is a schematic diagram of the cell-specific laser therapy catheter according to a diffusing fiber embodiment of the present invention;

FIG. 10 is a schematic diagram of the cell-specific laser therapy catheter according to an image-guided catheter embodiment of the present invention;

FIG. 11 is a flow chart of an exemplary embodiment of a technique for the cell specific therapy in arteries according to the present invention;

FIG. 12 is a flow diagram of an exemplary embodiment of an exogenous therapy method according to the present invention; and

FIG. 13 a flow diagram of an exemplary embodiment of a general cell specific therapy method according to the present invention that may utilize image guidance to determine the target location for therapy and or determine when the therapy has completed.

Throughout the figures, the same reference numerals and characters, unless otherwise stated, are used to denote like features, elements, components or portions of the illustrated embodiments. Moreover, while the subject invention will now be described in detail with reference to the figures, it is done so in connection with the illustrative embodiments. It is intended that changes and modifications can be made to the described embodiments without departing from the true scope and spirit of the subject invention as defined by the appended claims.

### DETAILED DESCRIPTION OF EXEMPLARY EMBODIMENTS

A chromophore is a molecule that absorbs light. In the absence of photochemical effects, the absorption of light can cause heating. An endogeous chromophore is a biological light absorbing molecule that is intrinsic to or resident within the cell. These molecules may absorb photons 200 which can heat the cell. Certain examples of endogenous chromophores can be water molecules, lipid, cell pigments etc. Cell death and irreversible protein denaturation can occur at temperatures above 6070° C. to 70° C. as described in A. L. McKenzie, “Physics of thermal processes in laser-tissue interaction,” Phys Med Biol. 1990;35, pp. 1175-209. In the absence of photochemical or vaporization processes, the energy absorbed by tissues in response to laser irradiation may be converted to heat, as described in A. Vogel et al., “Mechanisms of pulsed laser ablation of biological tissues,” Chem Rev. 2003;103, pp. 577-644. When energy is absorbed, heat transfer from the target to its cooler surroundings occurs by thermal diffusion.

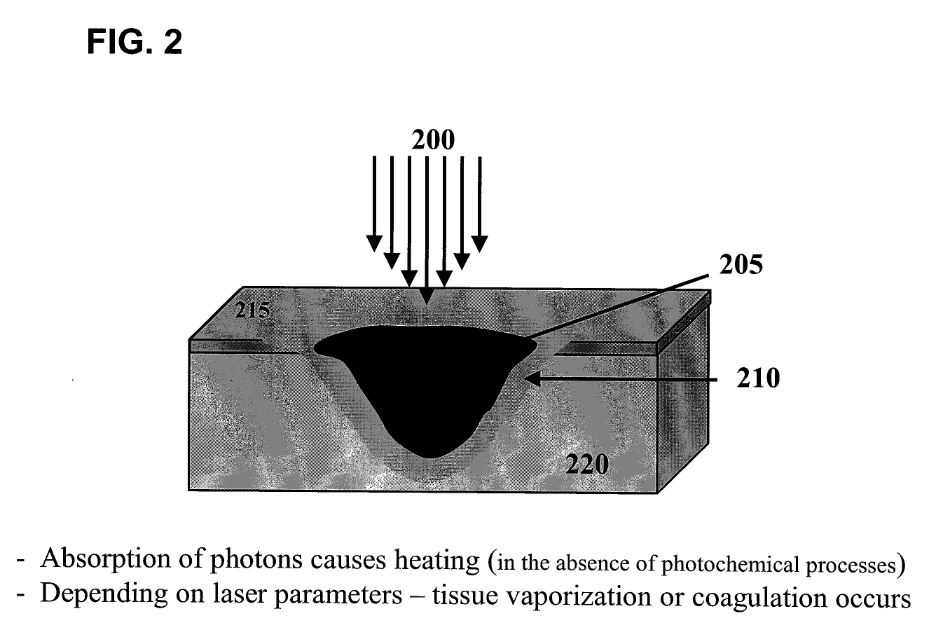

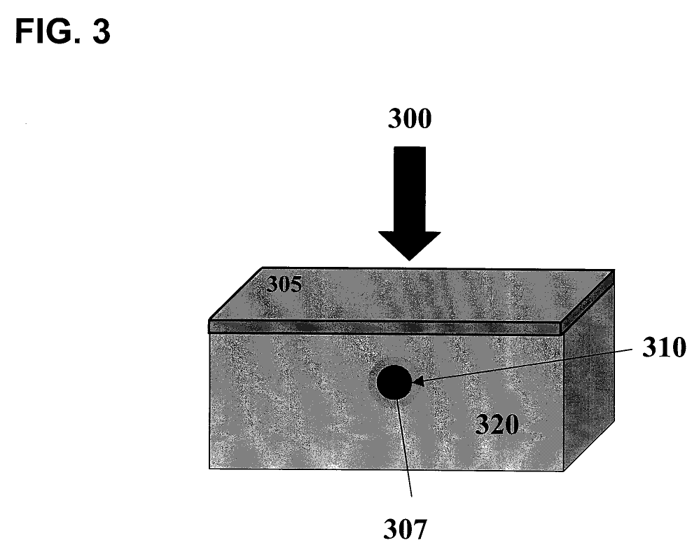

Depending on the laser parameters, different thermally induced effects can occur. When temperatures of 60° C. are reached, coagulation and irreversible denaturation of proteins 205 may occur which can causing cell death. At high temperatures over 100° C., vaporization likely occurs. When energy is absorbed, it can be spatially redistributed by thermal diffusion. The time it may take for this energy to be conducted can depend on the thermal relaxation time of the target chromophores. Depending on laser parameters a zone of thermal damage 205, 210, and 307 and 310 (shown in FIGS. 2 and 3, respectively) may occur around the region of laser ablation and the absorbed energy may be spatially redistributed by thermal conduction, as shown in FIG. 2. Based on these principles, previously, a technique labeled as Laser angioplasty was introduced to open up stenosed arteries. (See W. H. Ahmed et al., “Excimer laser coronary angioplasty,” Cardiol Clin. 1994;12, pp. 585-93). In this conventional method, the debulking of the plaque was performed by the laser ablation, and subsequent vaporization of the plaque in coronary arteries. Laser angioplasty resulted in high restenosis rates in patients possibly due to endothelial damage caused by the non-specfic tissue heating during laser ablation, initiating the process of SMC proliferation, neo-intimal thickening and restenosis.

### Selective Thermal Damage of Macrophages

Exemplary embodiment of the present invention relate to systems and methods for cell specific therapy by causing laser-induced thermal damage in atherosclerotic plaque macrophages by targeting endogenous (e.g., lipids) and exogenous (such as nanoparticles and microparticles) light absorbers. The sample approaches used according to exemplary embodiments of the present invention may not be to conduct ablation of the plaque as was previously done with the laser angioplasty, and to induce cell-specific thermal damage preferably only within macrophages, e.g., confining the zone of damage 307 and 310 (see FIG. 3), and thereby maintain the health of the endothelium and surrounding tissues 215, 220 (see FIG. 2) and tissues 320, 305 (see FIG. 3). An exemplary cell specific laser therapy may be performed as a stand alone procedure or in conjunction with OCT, OFDI, SD-OCT, Raman and IR spectroscopy, Laser Speckle Imaging (LSI), angioscopy, fluorescence, fluorescence spectroscopy, time resolved fluorescence, intravascular ultrasound (IVUS) systems/procedures, or any other imaging systems/procedures known in the art. The exemplary embodiment of the present invention can be associated with an observation that the optimized selection of laser parameters may be used to cause a cell specific thermal damage.

Previously, a concept of selective photothermolysis to achieve spatially confined localization of heat in tissues has been described. Using this exemplary method, selective thermal damage can be induced when the wavelength of the laser 300 (shown in FIG. 3) may be preferentially absorbed by the target chromophore 307, the required or preferable fluence is high enough to heat the chromophore, and the pulse duration of the laser exposure is shorter than the thermal relaxation time of the chromophore. (See R. R. Anderson et al., “Selective photothermolysis: precise microsurgery by selective absorption of pulsed radiation,” Science. 1983;220, pp. 524-7). The pulse duration, (t_{d}), of the exposure can influence the specificity or confinement of thermal damage, and may be determined from the thermal relaxation time (tᵣ) of the target chromophore. The transition from specific to non-specific thermal damage can occur when the ratio is as follows: (t_{d/}tᵣ)≧1. (See See R. R. Anderson et al., “Selective photothermolysis: precise microsurgery by selective absorption of pulsed radiation,” Science. 1983;220, pp. 524-7). For spheres of diameter, d, and thermal diffusivity, κ, the thermal relaxation time can be provided by tᵣ=(d²/27K)).

To induce targeted thermal damage in specific cells while maintaining the integrity of the surrounding tissue, it is preferable is that the target chromophores have greater optical absorption at a given laser wavelength than their surrounding tissue. According to an exemplary embodiment of the present invention, a thermal confinement within specific cell populations may be achieved by targeting various endogenous absorbers present in macrophages such as lipid droplets and cholesterol esters as light absorbers. (See C. M. Pitsillides et al., “Selective cell targeting with light-absorbing microparticles and nanoparticles,” Biophys J. 2003;84, pp. 4023-32).

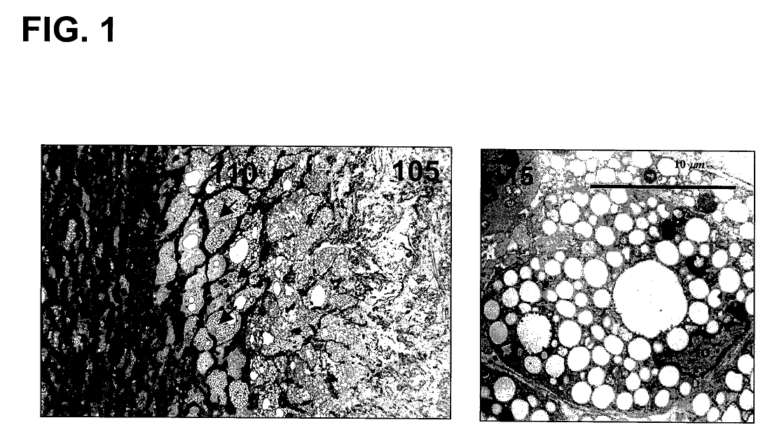

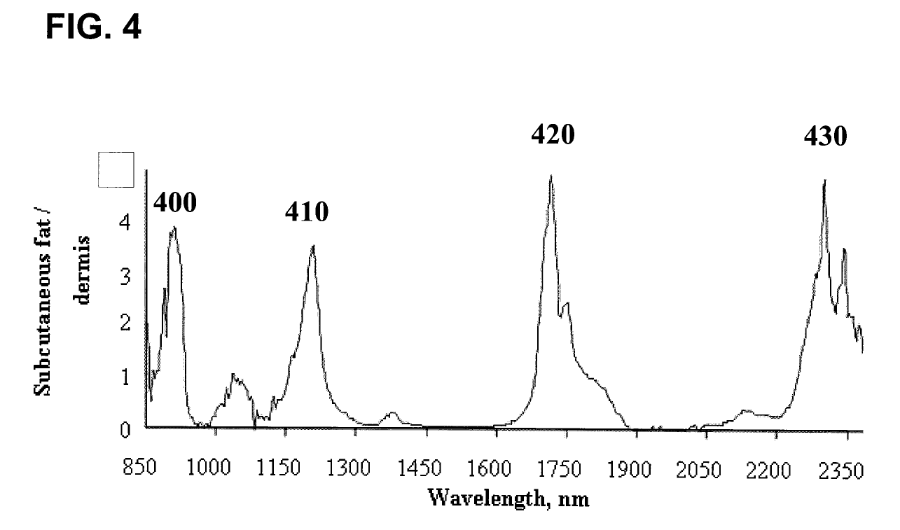

As shown in FIG. 1, plaque macrophages 105 may contain an abundance of lipid, which may provide an endogenous chromophore for selective heating and destruction of these cells while maintaining viability of the surrounding supportive cells and matrix. By targeting an endogenous absorber such as lipid, cholesterol or cholesterol esters, selective thermal damage can be induced in plaque macrophages. According to certain exemplary embodiments of the present invention, the targeting of endogenous chromophores for inducing macrophage cell death can preclude the requirement or preference for administering exogenous agents or chromophores. Confined energy deposition in lipid laden macrophages can be achieved by using laser energy at a wavelength that may be strongly absorbed by lipid and not by the surrounding aqueous tissue, and with a laser pulse duration that can be less than t, to reduce heat transfer from the absorbing lipid rich macrophages. By using spectro-photometric measurements, as shown in the exemplary graph of FIG. 4, at 915 nm (400), 1205 nm (410), 1715 nm (420) and 2305 nm (430) in the near and mid IR spectrum, lipid rich tissue may have a higher absorption than aqueous tissue. (See D. Manstein et al., “Selective photothermolysis of lipid rich tissue,” Annual meeting of Lasers in Surgery and Medicine. 2001; Supplement 13:Abs no 17). It may be possible to utilize a 1206 nm laser to induce selective thermal damage in subcutaneous fat, while maintaining the health of the overlying epidermis. In this exemplary embodiment of the present invention, laser wavelengths in the vicinity of absorption bands of endogenous chomophores, such as low density lipoprotein (LDL), free cholesterol, cholesterol esters, etc., may be used for inducing selective thermal damage of plaque macrophages.

### Demonstration of Cell-Specific Therapy of Macrophages

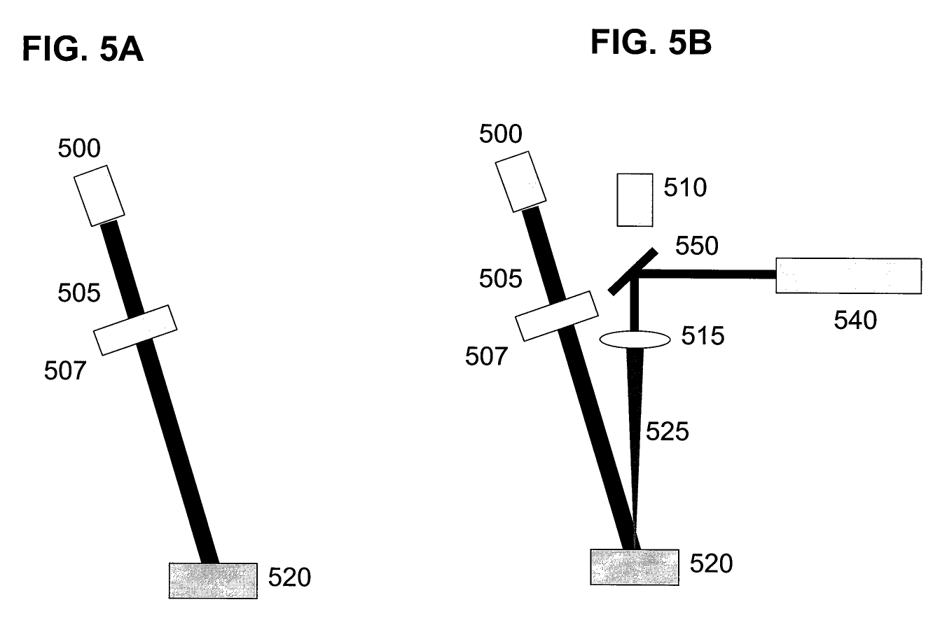

In one exemplary embodiment for achieving the goal of cell specific laser therapy according to the present invention can include an exemplary treatment delivery system to enable selective thermal confinement, as shown in FIG. 5A. For example, a laser source 510 having wavelengths in the vicinity of the absorption bands of target chromophores (e.g., lipid, cholesterol, cholesterol esters, etc.) within macrophages can be utilized for cell specific therapy. An output 505 of the laser 510 can be controlled to achieve macrophage cell death. To perform a laser-induced thermal confinement, the laser source can be configured to illuminate the tissue 520. The laser source 510 can be configured to permit pulsed operation by incorporating optical shutter, acousto-optical modulators 507 to facilitate a delivery of short laser pulses. The laser pulse duration, (t_{d}), can be adjusted such that the ratio, (t_{d/}tᵣ)≦1, where tᵣ is the thermal relaxation time.

According to one exemplary exemplary embodiment of the present invention, a 100 μm region comprising of lipid-filled macrophages can be used, and tᵣ may be approximately equal to 2 ms. Pulsed laser systems which can permit shorter pulse durations (˜10 μs) can provide for the targeting of single macrophages (e.g., tᵣ=20 μs). This exemplary process can be monitored by a direct thermal visualization using an exemplary thermal camera or other measurement device 510, or by another diagnostic imaging technique/procedure such as laser speckle imaging or optical frequency domain imaging. For example, as shown in FIG. 5B, a visible aiming beam from a Helium-Neon (632 nm) source 540 can be utilized to coincide with a center of the collimated treatment laser beam, as shown in FIG. 5. Laser speckle can be recorded using, e.g., a lens 515 and camera 510. A temporal modulation of the speckle pattern may be correlated to the temperature of the tissue 520 undergoing an exemplary selective laser ablation.

An exemplary identification of an appropriate wavelengths and exposure times for selective laser ablation can be determined using, e.g., cell culture experiments. Cell cultures can be conducted to evaluate laser induced thermal damage of lipid rich macrophages. Macrophage cells can be cultured in 75 ml flasks with DMEM, 0.1 M HEPES, 1% Penicillin Streptomycin and 10% fetal calf serum, and incubated at 10% CO₂. The cells can be cultured until they reach confluence and then scraped off the flasks with cell scrapers. The cells can be cultured with a 1 in 10 dilution in fresh medium to prevent macrophage activation. The cells can be counted with a hemocytometer and transferred to ten 6 well culture plates. For example, in five plates, low density lipoprotein labeled with FITC (e.g., Molecular Probes, Eugene, Oreg.) can be added followed by incubation for, e.g., up to 4 days.

The uptake of fluorescently-labeled LDL by macrophages can be assessed by fluorescence microscopy. It is possible to utilize, e.g., two control cell populations for the review: a) macrophages not be incubated with LDL, and b) human coronary smooth muscle cell lines (HCASMC-c) can be grown in culture and not incubated with LDL. The macrophage and control cell culture plates can be exposed to laser irradiation using the pulsed laser ablation system described above. Laser therapy can be conducted by scanning a focused laser beam or illuminating a large area using a collimated laser beam. The laser pulse duration and number of pulses can be varied to evaluate the influence of these parameters on laser induced cell necrosis. After the exemplary laser treatment, cell viability assays (such as propiodine iodide) can be used to evaluate cell death following thermal damage. The cells can be assessed using microscopy and the percentage of cell death can be quantified by cell counting using a flow cytometer. The percentage of cell death in the LDL ingested macrophage population can be compared with the control cell populations. The correlation of percent cell death with laser exposure parameters may be evaluated using the regression analysis.

Freshly harvested samples of human carotid, coronary, iliac and aortic arteries obtained at autopsy can be used to evaluate the cell specificity of thermal confinement. The specimens can be opened longitudinally and pinned to expose the luminal side. The tissue specimens can be then irradiated using the pulsed laser ablation system described above. The laser pulse duration and number of pulses can be varied to evaluate the influence of these parameters on thermal confinement within macrophage rich regions in atherosclerotic plaques. Following treatment laser exposure, the specimens can then be grossly sectioned and prepared for histological processing. The specimes can be stained using Hematoxylin Eosin, and CD68 for macrophages. Nitro blue tetrazolium chloride (NBTC) staining can be used to assess the extent of thermal damage. NBTC stains positive for lactate dehydrogenase (LDH), which is a thermolabile enzyme. A loss of LDH activity ensues rapidly upon heat induced cell damage and is correlated with cell lethality. (See M. H. Khan et al., “Intradermally focused infrared laser pulses: thermal effects at defined tissue depths,” Lasers Surg Med. 2005;36, pp. 270-280).

The region of the border between unstained and stained tissue can be morphometrically measured to evaluate the area of thermal damage. The CD68 stained sections can be co-registered with NBTC stained sections to evaluate cell specificity of laser treatment. A metric for cell specificity of necrosis can be estimated by measuring the ratio between the areas of CD 68 staining to loss of LDH staining. The correlation of the cell specificity metric with laser pulse duration and number of pulses can be determined. The optimum laser parameters that achieve a cell specificity metric close to unity can be determined. Thus, the optimal treatment laser parameters for subsequent development of an intracoronary real-time screening and therapy device can be determined that may identify macrophage-rich regions and selectively destroy them with laser energy. This exemplary development can fill the needed gap between the detection of unstable plaque and local therapy of these lesions. Such exemplary embodiments may furthermore provide the foundation for cell specific laser therapy in a variety of other diseases, where endogenous absorbers can be targeted to effect selective damage of the abnormal cells while maintaining the viability of surrounding normal cells.

### Cell Specific Therapy by Administration of Exogenous Nanoparticles

Another exemplary embodiment of the system and method according to the present invention can include the administration of exogenous metal or noble metal nanoparticles via subcutaneous, oral or intravenous arrangement. The nanoparticles of appropriate size, e.g., preferably <about 5 nm, may penetrate the vascular endothelium and can be taken up by macrophages resident in the tissue of interest. These nanoparticles can be capable of then being irradiated by light. Direct absorption or surface plasmon resonance associated with these nanoparticles, can cause local and specific heating that will thermally damage the cells containing the nanoparticles. According to yet another exemplary embodiment of the present invention, these nanoparticles may be imaged by techniques including but not limited to those mentioned in this document, in such a manner as to determine the appropriate locations for administration of selective laser therapy light.

Still another exemplary exemplary embodiment of the system and method according to the present invention can be provided for image-guided cell-specific laser therapy. In these exemplary system and method, high-resolution volumetric screening of tissue can be conducted using imaging techniques such as Optical Frequency Domain Imaging (OFDI) to detect tissue macrophages and enable simultaneous guidance of therapeutic laser irradiation to induce macrophage cell death by probing exogenous chromophores phagocytosed by macrophages. The exemplary OFDI techniques, systems and procedures can be used for comprehensive volumetric screening of tissue which enables the identification of tissue macrophages in situ. (See, e.g., B. D. MacNeill et al., “Focal and multi-focal plaque macrophage distributions in patients with acute and stable presentations of coronary artery disease,” J Am Coll Cardiol. 2004;44, pp. 972-9. Exemplary system, catheter and method according to the present invention can be provided for simultaneous macrophage detection and delivery of therapeutic laser energy. Exogenous chromophores administered can include nobel metal nanoparticles, biodegradable nanoparticles or iron oxide microparticles to cause laser induced thermal confinement within macrophages while maintaining the health of the surrounding tissue.

An exemplary emboidment of a laser treatment system and method can be provided that may utilize a laser source configured with an acouto-optic modulator to permit pulsed operation to enable thermal confinement. The wavelength of the light source can be provided depending on the chromophore under investigation. Macrophage cells (J774 cell line) may be cultured in 75 ml flasks using a growth medium. To mimic plaque macrophages, the cultured cells can be separately incubated for, e.g., up to four days with fluorescently labeled low density lipoprotein (LDL) (e.g., Molecular Probes, Eugene, Oreg.). The uptake of LDL may be evaluated using fluorescence microscopy.

As for endogenous selective cell therapy, the proper exposure and power preferences for effecting cell damage may determined using cell culture studies. A population of LDL ingested macrophages will be separately incubated with nobel metal nanoparticles, biodegradable nanoparticles or iron oxide microparticles tuned to the treatment laser wavelength. For example, two control cell populations can be used: i) macrophages that would not be incubated with LDL or exogenous chromophores, and ii) human coronary smooth muscle cell lines (HCASMC-c) may be grown in culture and not incubated with LDL or nanoparticles. Most or all culture plates can be exposed to laser irradiation and the percentage of cell death may be quantified using propidium iodide assays. The laser pulse duration, incident power and number of pulses will be varied to evaluate the influence of these parameters on laser induced cell death in all cell populations.

Animal studies may also be utilized to determine biodistribution of nanoparticles as well as proper exposure and power parameters for cell specific laser therapy. According to one exemplary embodiment of the present invention, it may be preferable to determine the distribution of gold, silver and USPIO nanoparticles in hyperlipidemic animal atherosclerotic plaques. For example, each of the three nanoparticles, gold, silver and USPIOs can be tested independently using a similar study design. This can be done for each nanoparticle by a daily intravenous administration of the agent to, e.g., 15 Watanabe heritable hyperlipidemic (WHHL) rabbits (see P. M. McCabe et al. “Social environment influences the progression of atherosclerosis in the watanabe heritable hyperlipidemic rabbit,” Circulation, 2002;105, pp. 354-9; S. Ojio et al. “Considerable time from the onset of plaque rupture and/or thrombi until the onset of acute myocardial infarction in humans: coronary angiographic findings within 1 week before the onset of infarction,” Circulation, 2000;102, pp. 2063-9; and K. Yokoya et al. “Process of progression of coronary artery lesions from mild or moderate stenosis to moderate or severe stenosis: A study based on four serial coronary arteriograms per year,” Circulation, 1999;100, pp. 903-9), which may develop active aortic plaques at 6 months of age, and to 5 New Zealand White (NZW) rabbit that will act as a control for each agent. Five additional WHHL rabbits can be investigated without the administration of any agent to provide diseased controls for each group.

For example, most or all rabbits can be approximately one-year-old. Nanoparticle agents may be administered daily for up to 5 days through auricular veins during sedation with isoflurane (1%), at doses of 1-2 mg/kg. WHHL rabbits receiving nanoparticle agents will be euthanized on days 2, 3, and 4 (3 rabbits per time point). The control rabbits can be euthanized on day 4. Perfusion fixation will be performed prior to aortic harvest. Serial histological sections will be cut at 5 microns and stained with hematoxylin-eosin, Masson's Trichrome, CD 68 immunoperoxidase and Prussian blue. The patterns of nanoparticle distribution may be correlated with histological determinants of plaque vulnerability, namely lipid core and cap 100 thickness. Further, 2 mm sections of each aortic specimen will be subjected to electron microscopic evaluation to determine precisely the intracellular site and ultrastructural morphology and intracellular distribution of nanoparticle deposition.

According to another exemplary embodiment of the present invention, it may be preferable to measure the optical signature associated with nanoparticle uptake in hyperlipidemic animal atherosclerotic plaques. For example, after euthanization and prior to perfusion fixation of the rabbit aortas described above, optical coherence tomography (OCT) technique(s) and angioscopic imaging may be conducted using automatic pullback at a rate of 0.5 mm/second from the iliac artery to the aortic arch. The aortas may then be opened and reflectance confocal microscopy will be conducted along the length of each vessel. For each agent and time point, images obtained from the treated rabbits can be morphometrically and spectroscopically compared to images acquired from the control rabbits. A quantitative analysis of signal intensities in regions of interest within the plaque, including the cap shoulder, the body of the cap and the lipid rich core may be assessed to evaluate the quantitative distribution of each agent within atherosclerotic plaque. Tissue locations with unique optical signatures relative to control rabbit measurements will be selectively taken and processed for histology and electron microscopy.

According to still another exemplary embodiment of the present invention, it may be preferable to demonstrate a quantification of nanoparticle-labeled macrophages in vivo. The nanoparticle metal achieving the greatest optical contrast can be administered to 10 WHHL rabbits (1-year-old) at a dose of 2 mg/kg. For example, five additional WHHL rabbits not receiving the nanoparticle agent may be used as controls. On the optimal day following nanoparticle administration (determined as provided above), exemplary OCT imaging technique can be performed and the rabbits may be sacrificed. For example, IM injection of ketamine (35 mg/kg)/xylazine(7 mg/kg) can be administered with local anesthesia (lidocaine) in the inguinal region.

A continuous assessment of the rabbits response to corneal and jaw reflexes will be used to monitor the level of anesthesia. The left iliac artery may be exposed and isolated via a cutdown procedure. A 6F introducer may be placed in the left iliac artery. A 0.014″ guidewire can be advanced into the aorta. Under fluoroscopic guidance, the OCT catheter (3F) may be advanced through the introducer, over the guidewire and into the aorta. The exemplary OCT imaging of the aorta and iliac arteries may be performed using anatomic landmarks for image registration. After imaging, the animals may be sacrificed. Histologic sections can be taken from plaques adjacent to the anatomic landmarks identified while imaging with OCT under fluoroscopic guidance. The tissue can be processed in a routine fashion. Four-micron sections may be cut at the OCT imaging sites and stained with hematoxylin and eosin (H&E) and Masson's trichrome. To visualize the presence of macrophages, a mouse-antirabbit CD68 monoclonal antibody may be used (Dako Corporation).

Immunohistochemical detection of the preferred epitopes can be performed according to the indirect horseradish peroxidase technique. Using both digitized histology and OCT techniques, measurements of macrophage density may be obtained using a 500×125 μm (lateral×axial) region of interest (ROI), located in the center of each plaque. The area percentage of CD68+ staining can be quantified (at 100× magnification) using automatic bimodal color segmentation within the corresponding ROI's of the digitized immunohistochemically stained slides. The OCT signal intensity and standard deviation within each plaque may then be compared with immunohistochemical staining from slides obtained from corresponding locations using linear regression.

### Image-Guided Cell Specific Laser Therapy

Cell specific laser therapy may be conducted as a standalone technique/procedure or in conjunction with imaging or spectroscopic techniques/procedures for diagnosis for target atherosclerotic plaques and guidance of therapy. Techniques such as Laser Speckle imaging (e.g., as shown in FIG. 5), angioscopy, fluorescence, fluorescence spectroscopy, time-resolved fluorescence, OCT, OFDI, SDOCT, Raman or IR spectroscopy, IVUS, intra-vascular MRI etc may be used to detect culprit plaques and guide cell specific laser therapy. One exemplary embodiment for image-guided cell specific therapy involves the use of OCT and/or next generation OCT methods such as OFDI or spectral-domain OCT (SD-OCT) for detection of macrophage rich plaques to target therapy.

### Catheters for Cell Specific Laser Therapy

### i. Single Exemplary Optical Fiber Embodiment

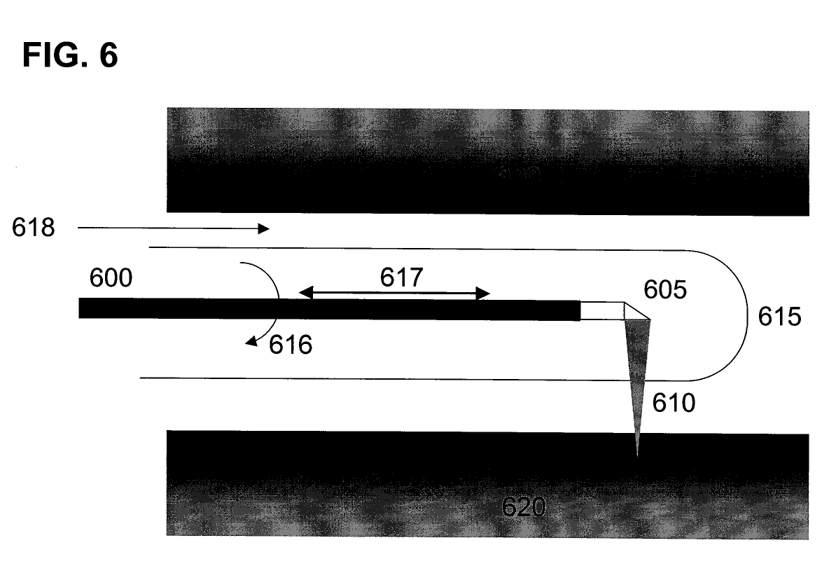

This exemplary embodiment can include a design for a standalone approach for comprehensive cell specific laser therapy without the use of image guidance (FIG. 6). In this exemplary embodiment, light from the pulsed laser source can be coupled to the proximal end of an optical fiber 600. The optical fiber can be housed in an outer sheath 615. The fiber can be terminated by beam focusing and/or beam redirecting optics 605 to direct and focus the light 610 at a pre-determined location on the artery wall. Laser light at the distal end can be collimated or focused by a lens, which can be a micro-lens, GRIN lens or the like. The fiber can be configured to scan the beam in at least one of a rotational 616 or longitudinal 617 or another direction along the vessel wall 620. For this embodiment, therapy can be conducted with flushing the vessel lumen 618 in order to maintain good beam quality and avoid scattering and absorption of therapy light by blood.

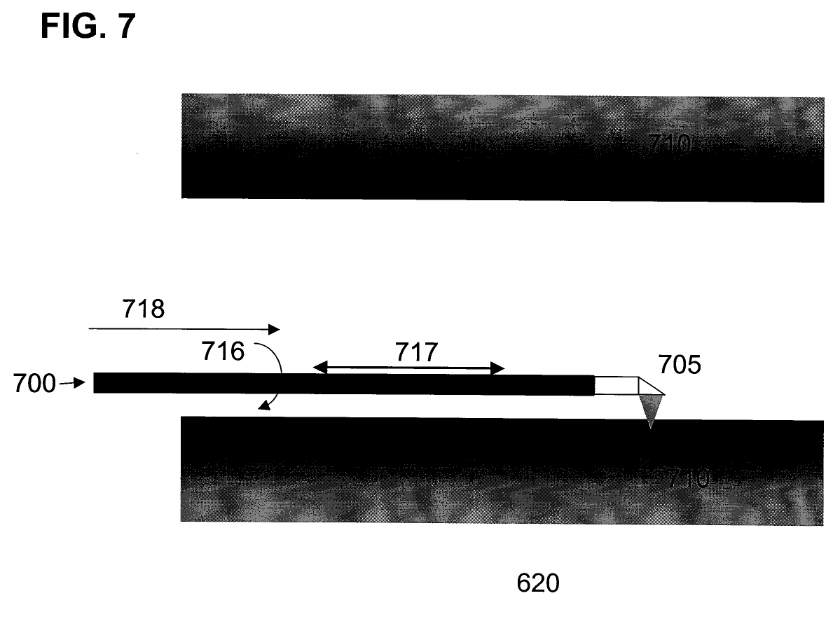

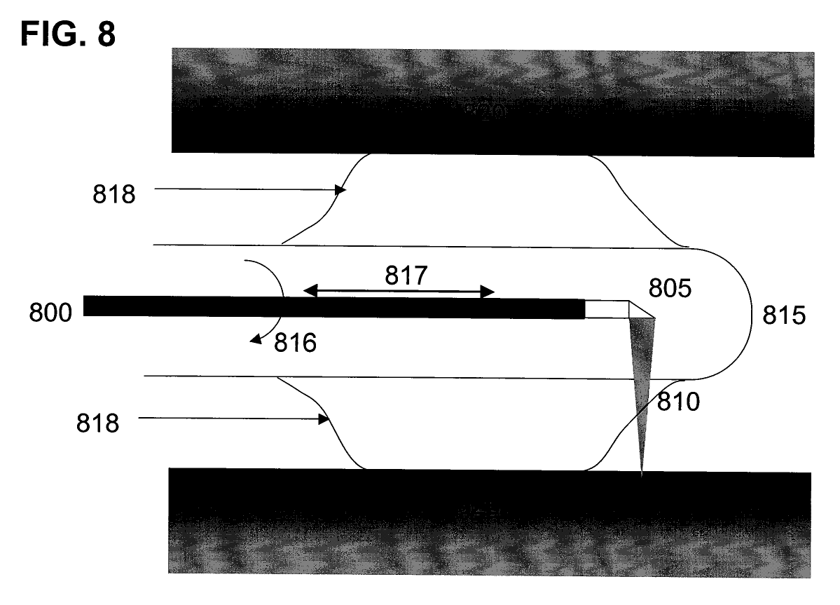

In another exemplary embodiment as shown in FIG. 7, the therapy fiber 700 can be configured to contact or be near contact to the vessel wall 710. The fiber can be scanned in at least one of a rotational 716 or longitudinal 717 or other direction to treat a segment of the artery. In still another exemplary embodiment illustrated in FIG. 8, the therapy fiber can reside within a balloon 818. The balloon 818 can be inflated in the area of the vessel wall 820 requiring treatment, and the fiber 800 can scan in at least one of a rotational direction 816, longitudinal direction 817 or another direction to treat the area of interest.

### ii. Diffusing Catheter Exemplary Embodiment

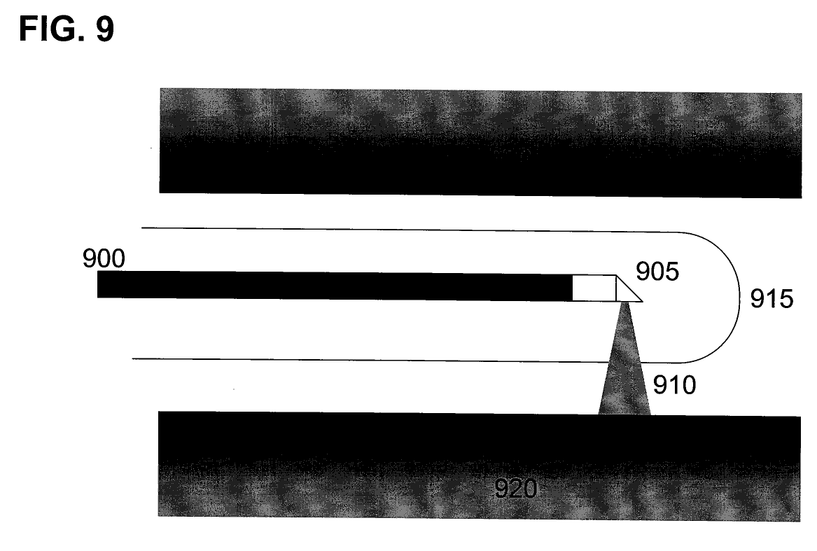

According to a further exemplary embodiment of the present invention as shown in FIG. 9, the light 910 associated with a target chromophore can be diffused over a large area of the artery wall 920 using a balloon, diffusing optic 905 or the like.

### iii. Vascular Cell-Specific Laser Therapy with Cooling

Even though the specificity of the chromophore may allow selective destruction of the cells of interest, if the absorption coefficient differential between target and surrounding tissue is not large enough, collateral damage at the surface of the tissue may occur, resulting in damaged endothelium. In order to avoid this possibly untoward effect, the surface of the endothelium may be cooled by use of cooled saline, water, D₂O, blood or other cooled liquid during the therapy laser irradiation. This procedure can maintain viability of endothelium while safely applying cell-specific laser irradiation deeper into the vessel wall. Therefore, the catheter can be associated with a mechanism for flushing the vessel with coolant. In one exemplary embodiment of the present invention, this mechanism can include a guide catheter that may contain the therapy catheter therein. In another exemplary embodiment, the therapy catheter may contain a flushing port. In still another embodiment, the catheter can contain a balloon, which may be filled with said coolant.

### iv. Image-Guided Exemplary Catheter Embodiments

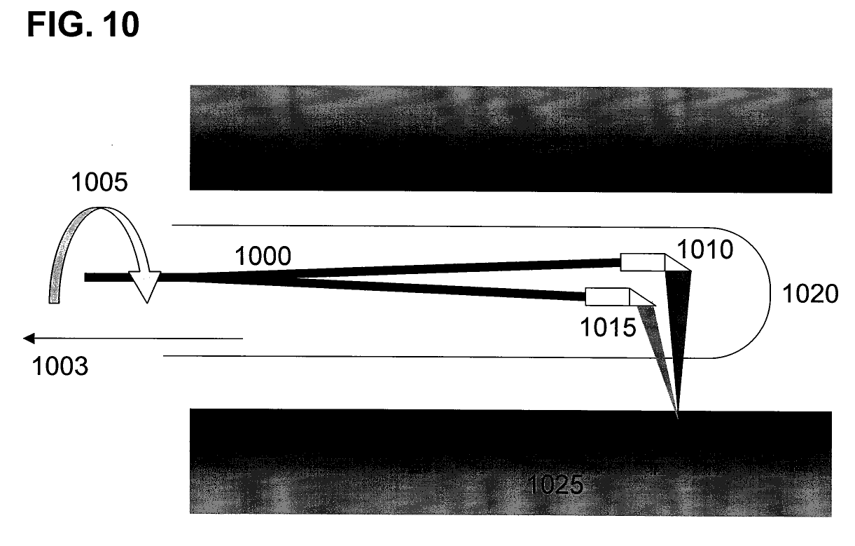

This exemplary embodiment of the present invention can include a probe design for comprehensive volumetric diagnosis and screening for target atherosclerotic plaque and simultaneous cell specific laser therapy of macrophages in atherosclerotic plaques, as shown in FIG. 10. The exemplary probe 1000 illustrated in FIG. 10 can be configured to scan across the luminal surface of the artery in at least one of an axial direction 1003, a radial direction 1005 or another direction. Therapeutic laser light 1015 and optical diagnostic arrangement 1010 (e.g., Laser Speckle imaging, angioscopy, OCT, OFDI, SDOCT, Raman or IR spectroscopy, fluorescence, fluorescence spectroscopy, time-resolved fluorescence arrangement) beams can be delivered through the same or separate optical fibers. For distinct diagnosis and therapy fibers, each optical fiber may have its own distal optics to produce its own optical diagnosis and therapy beam diameters on the target tissue. In one embodiment, the optical fibers and distal optics are housed in a drive shaft and placed inside a catheter sheath 1020. The proximal end of the catheter may be coupled to a rotary junction and mounted on a motorized pull back unit. Rotation of the inner components of the catheter and pullback will enable simultaneous diagnosis and therapy. In another exemplary embodiment of the present invention, the diagnosis and therapy catheters can be configured to be in contact with the artery wall 1025. The fibers can be scanned along the endothelium to diagnose and provide cell-specific therapy at the contact point.

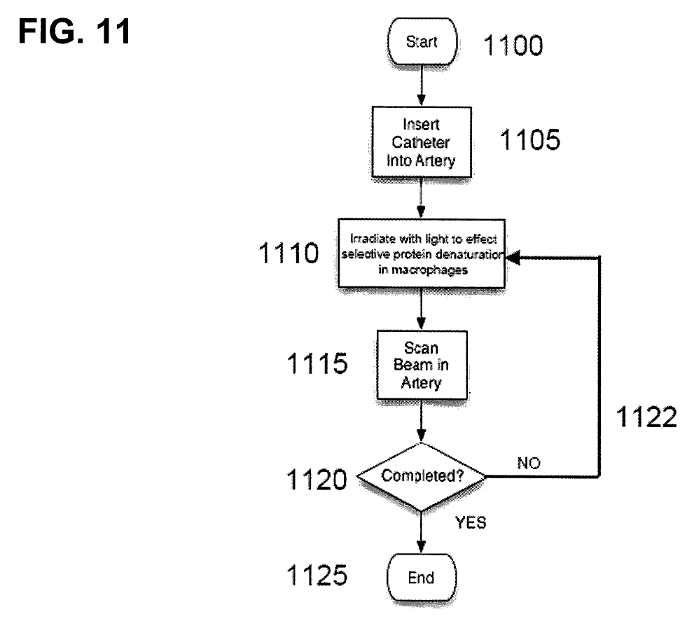

Certain exemplary embodiments for cell specific therapy methods according to the present invention are described below and shown in exemplary flow diagrams of FIGS. 11-13. For example, FIG. 11 shows an exemplary flow diagram of an exemplary embodiment of an endogenous therapy method according to the present invention, whereby the catheter can be started (step 1100) and inserted in to an artery (step 1105) and laser irradiation with appropriate wavelength, exposure parameters, and power density may be directed into the artery (step 1110). The wavelength may be selected to obtain a large differential absorption between plaque macrophages and surrounding tissues. The exposure and power parameters can be selected to affect thermal confinement to these cells. Blood may or may not be removed prior to laser irradiation. The artery is exposed by the light for a pre-determined time. The catheter may move in at least one of a circumferential, longitudinal or other direction (step 1115) to scan the entire treatment area. Alternatively or in addition, the light may diffuse throughout the artery without beam scanning. In step 1120, it may be determined whether the exemplary procedure is completed, and if not, the procedure may be repeated (step 1122).

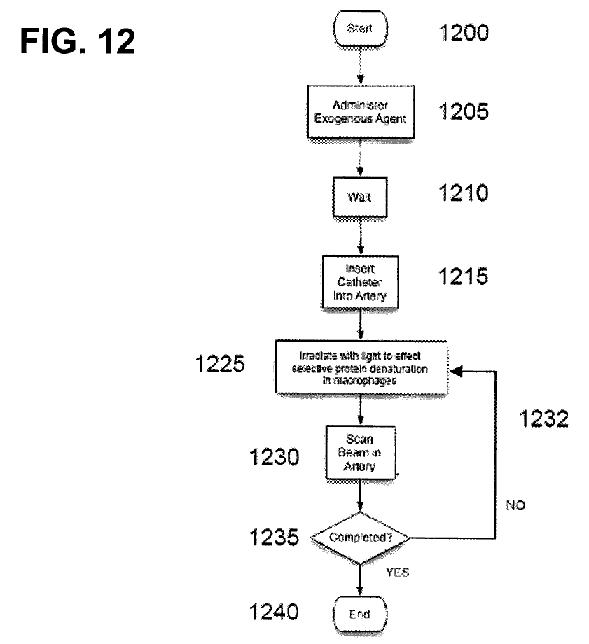

FIG. 12 depicts a flow diagram of an exemplary embodiment of an exogenous therapy method according to the present invention, whereby the procedure is started in step 1200, and an exogenous substance such as noble metal nanoparticles may be administered to the patient (step 1205). Following an appropriate time period (step 1210), the catheter is inserted into an artery (step 1215), and laser irradiation with appropriate wavelength, exposure parameters, and power density may be directed into the artery (step 1225). The wavelength is selected to obtain a large differential absorption between plaque macrophages and surrounding tissues. The exposure and power parameters are selected to affect thermal confinement to these cells. Blood may or may not be removed prior to the laser irradiation. The artery is exposed to therapy light for a pre-determined time. The catheter may move in at least one of a circumferential, longitudinal or other direction (step 1230) to scan the entire treatment area. Alternatively or in addition, the therapy light may be diffused throughout the artery without scanning. The procedure may be repeated (step 1232).

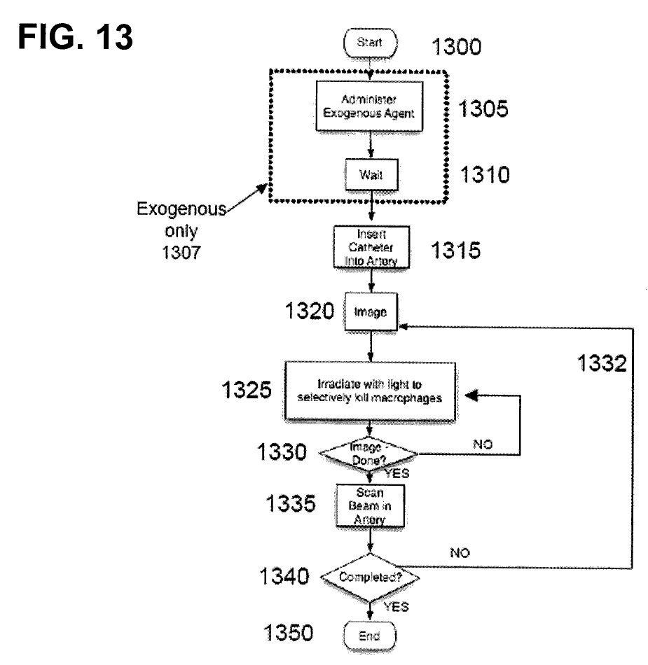

FIG. 13 shows a flow diagram of an exemplary embodiment of a general cell specific therapy method according to the present invention that may utilize image guidance to determine the target location for therapy and or determine when the therapy has completed. This exemplary method can be implemented for endogenous absorbers (e.g., without elements provided in box 1307) or exogenous absorbers (e.g., with elements provided in box 1307). The procedure may be started in step 1300, the exogenous agent can be administered in step 1305, and then it is possible to wait (step 1310). The catheter may be inserted into an artery (step 1315) and information can be retrieved from the artery wall to determine if therapeutic laser irradiation should be deployed (step 1320).

At appropriate exemplary locations, e.g., determined by an exemplary diagnostic method, the light irradiates the wall with appropriate wavelength, exposure parameters, and power density (step 1325). The exposure and power parameters are selected to affect thermal confinement to these cells. The artery may be exposed to therapy light for a pre-determined time or a time determined by feedback to the same or another exemplary diagnostic method (step 1330). The catheter may move in at least one of a circumferential, longitudinal or other direction to scan the entire treatment area (step 1335). The procedure may be repeated (step 1332).

### EXEMPLARY REFERENCES

1. Chambers A F, Naumov G N, Varghese H J, Nadkarni K V, MacDonald I C, Groom A C. Critical steps in hematogenous metastasis: an overview. Surg Oncol Clin N Am. 2001;10:243-55, vii.
2. Chambers A F. The metastatic process: basic research and clinical implications. Oncol Res. 1999;11:161-8.
3. Tabas I. Consequences and therapeutic implications of macrophage apoptosis in atherosclerosis: the importance of lesion stage and phagocytic efficiency. Arterioscler Thromb Vasc Biol. 2005;25:2255-64.
4. Brinkmann R, Huttmann G, Rogener J, Roider J, Birngruber R, Lin C P. Origin of retinal pigment epithelium cell damage by pulsed laser irradiance in the nanosecond to microsecond time regimen. Lasers Surg Med. 2000;27:451-64.
5. Manstein D, Erofeev A V, Altshuler G B, Anderson R R. Selective photothermolysis of lipid rich tissue. Annual meeting of Lasers in Surgery and Medicine. 2001;Supplement 13:Abs no 17.
6. Anderson R R, Margolis R J, Watenabe S, Flotte T, Hruza G J, Dover J S. Selective photothermolysis of cutaneous pigmentation by Q-switched Nd: YAG laser pulses at 1064, 532, and 355 nm. J Invest Dermatol. 1989;93:28-32.
7. Del Governatore M, Hamblin M R, Shea C R, Rizvi I, Molpus K G, Tanabe K K, Hasan T. Experimental photoimmunotherapy of hepatic metastases of colorectal cancer with a 17.1A chlorin(e6) immunoconjugate. Cancer Res. 2000;60:4200-5.
8. Hayase M, Woodbum K W, Perlroth J, Miller R A, Baumgardner W, Yock P G, Yeung A. Photoangioplasty with local motexafin lutetium delivery reduces macrophages in a rabbit post-balloon injury model. Cardiovasc Res. 2001;49:449-55.
9. Whitesides G M. The ‘right’ size in nanobiotechnology. Nat Biotechnol. 2003;21:1161-5.
10. Weissleder R, Kelly K, Sun E Y, Shtatland T, Josephson L. Cell-specific targeting of nanoparticles by multivalent attachment of small molecules. Nat Biotechnol. 2005;23:1418-23.
11. Tsapis N, Bennett D, Jackson B, Weitz D A, Edwards D A. Trojan particles: large porous carriers of nanoparticles for drug delivery. Proc Natl Acad Sci USA. 2002;99:12001-5.
12. Pitsillides C M, Joe E K, Wei X, Anderson R R, Lin C P. Selective cell targeting with light-absorbing microparticles and nanoparticles. Biophys J. 2003;84:4023-32.
13. Ross R. Atherosclerosis—an inflammatory disease. N Engl J Med. 1999;340:115-26.
14. Libby P. Inflammation in atherosclerosis. Nature. 2002;420:868-74.
15. MacNeill B D, Jang I K, Bouma B E, Iftimia N, Takano M, Yabushita H, Shishkov M, Kauffman C R, Houser S L, Aretz H T, DeJoseph D, Halpern E F, Tearney G J. Focal and multi-focal plaque macrophage distributions in patients with acute and stable presentations of coronary artery disease. J Am Coll Cardiol. 2004;44:972-9.
16. Libby P, Aikawa M. Stabilization of atherosclerotic plaques: new mechanisms and clinical targets. Nat Med. 2002;8:1257-62.
17. McKenzie A L. Physics of thermal processes in laser-tissue interaction. Phys Med Biol. 1990;35:1175-209.
18. Vogel A, Venugopalan V. Mechanisms of pulsed laser ablation of biological tissues. Chem Rev. 2003;103:577-644.
19. Ahmed W H, Bittl J A. Excimer laser coronary angioplasty. Cardiol Clin. 1994;12:585-93.
20. Anderson R R, Parrish J A. Selective photothermolysis: precise microsurgery by selective absorption of pulsed radiation. Science. 1983;220:524-7.
21. Khan M H, Sink R K, Manstein D, Eimerl D, Anderson R R. Intradermally focused infrared laser pulses: thermal effects at defined tissue depths. Lasers Surg Med. 2005;36:270-280.
22. Yabushita H, Bouma B E, Houser S L, Aretz H T, Jang I K, Schlendorf K H, Kauffman C R, Shishkov M, Kang D H, Halpern E F, Tearney G J. Characterization of human atherosclerosis by optical coherence tomography. Circulation. 2002;106:1640-5.
23. Jang I K, Tearney G J, MacNeill B, Takano M, Moselewski F, Iftima N, Shishkov M, Houser S, Aretz H T, Halpern E F, Bouma B E. In vivo characterization of coronary atherosclerotic plaque by use of optical coherence tomography. Circulation. 2005;111:1551-5.
24. Tearney G J, Yabushita H, Houser S L, Aretz H T, Jang I K, Schlendorf K H, Kauffman C R, Shishkov M, Halpern E F, Bouma B E. Quantification of macrophage content in atherosclerotic plaques by optical coherence tomography. Circulation. 2003;107:113-9.

The foregoing merely illustrates the principles of the invention. Various modifications and alterations to the described embodiments will be apparent to those skilled in the art in view of the teachings herein. Indeed, the arrangements, systems and methods according to the exemplary embodiments of the present invention can be used with and/or implement any OCT system, OFDI system, SD-OCT system, Laser Speckle Imaging (LSI) systems or other imaging systems, and for example with those described in International Patent Application PCT/US2004/029148, filed Sep. 8, 2004, U.S. patent application Ser. No. 11/266,779, filed Nov. 2, 2005, U.S. patent application Ser. No. 10/501,276, filed Jul. 9, 2004, U.S. patent application Ser. No. 11/624,334 filed Jan. 18, 2007, and U.S. patent application Ser. No. 10/551,735 filed Sep. 29, 2005, the disclosures of which are incorporated by reference herein in their entireties. It will thus be appreciated that those skilled in the art will be able to devise numerous systems, arrangements and methods which, although not explicitly shown or described herein, embody the principles of the invention and are thus within the spirit and scope of the present invention. In addition, to the extent that the prior art knowledge has not been explicitly incorporated by reference herein above, it is explicitly being incorporated herein in its entirety. All publications referenced herein above are incorporated herein by reference in their entireties.

What is claimed is:

1. An apparatus comprising:
at least one arrangement configured to forward an electromagnetic radiation to an anatomical structure, the electromagnetic radiation having at least one property configured to:
i. modify at least one characteristic of at least one first cell, and
ii. at least one of (a) minimize any modification of or (b) modify at least one characteristic of at least second cell,
wherein the first and second cells are different from one another, wherein the characteristics of the first and second cells are different from one another, wherein at least one of the first cell or the second cell has at least one macrophage feature, and wherein the at least one characteristic of at least one of the at least one first cell or the at least one second cell is temperature.

2. The apparatus according to claim 1, wherein the at least one property includes at least one of wavelength, average power, instanteneous power, pulse duration or total exposure duration.

3. The apparatus according to claim 2, wherein the at least one property includes a wavelength which is approximately same as a wavelength of an absoption characteristic of a compound within the at least one first cell.

4. The apparatus according to claim 2, wherein the at least one property includes a pulse duration which causes the at least one characteristic to be be confined approximately within the at least one first cell.

5. The apparatus according to claim 2, wherein the at least one property includes power which causes the at least one characteristic to irreversably damage at least one portion of the at least one first cell.

6. The apparatus according to claim 1, wherein the at least one charactristic of the at least one first cell is greater than a temperature that causes a damage to at least one portion of the at least one first cell.

7. The apparatus according to claim 7, wherein the damage to the at least one portion of the at least one first cell is irreversible.

8. The apparatus according to claim 1, wherein the at least one first cell is situated within a vascular wall.

9. The apparatus according to claim 8, wherein the vascular wall is a part of a coronary artery.

10. The apparatus according to claim 1, wherein the at least one arrangement is provided in a catheter.

11. The apparatus according to claim 1, wherein the at least one macrophage feature is a lysozome containing at least one lipid.

12. The apparatus according to claim 11, wherein the at least one lipid includes at least one low density lipo protein (“LDL”), oxidized LDL, cholesterol or cholesterol ester.

13. The apparatus according to claim 1, wherein the at least one macrophage feature is a lysozome containing nacrotic debris.

14. The apparatus according to claim 1, wherein the at least one macrophage feature is a multitude of lysozomes.

15. The apparatus according to claim 1, wherein the at least one macrophage feature is at least one nanoparticle.

16. The apparatus according to claim 1, wherein the least one nanoparticle is provided within the cell.

17. The apparatus according to claim 16, wherein the least one nanoparticle is provided within a lysozome.

18. The apparatus according to claim 16, wherein a size of a single one of the least one nanoparticle is between 1 and 20 nanometer.

19. The apparatus according to claim 16, wherein the least one nanoparticle is comprised of at least one of a metal, nobel metal, ultra-small para-magnetic iron oxide, gold or silver.

20. The apparatus according to claim 16, wherein the least one nanoparticle is administered to a subject interveneously.

21. The apparatus according to claim 16, wherein, when the electromagnetic radiation is applied on the least one nanoparticle, a surface plasmon is generated.

22. The apparatus according to claim 1, wherein the at least one first arrangement is configured to forward an electromagnetic radiation externally from a body of a subject.

23. The apparatus according to claim 1, wherein the at least one macrophage feature provided within a fiberous cap.

24. The apparatus according to claim 1, further comprising at least one second arrangement configured to determine a location associated with at least one of the at least one first cell and the at least one second cell, wherein the at least one first arrangement is further configured to forward the electromagnetic radiation in a vicinity of the location.

25. The apparatus according to claim 24, wherein at least one of the first cell or the second cell has at least one macrophage feature.

26. The apparatus according to claim 24, wherein the at least one characteristic of at least one of the at least one first cell or the at least one second cell is temperature.

27. A method comprising:
forwarding an electromagnetic radiation to an anatomical structure, the electro-magnetic radiation having at least one property configured to:
iii. modify at least one characteristic of at least one first cell, and
iv. at least one of (a) minimize any modification of or (b) modify at least one characteristic of at least second cell,
wherein the first and second cells are different from one another, wherein the characteristics of the first and second cells are different from one another, wherein at least one of the first cell or the second cell has at least one macrophage feature, and wherein the at least one characteristic of at least one of the at least one first cell or the at least one second cell is temperature.

28. An apparatus comprising:
at least one first arrangement configured to forward an electromagnetic radiation to an anatomical structure, the electro-magnetic radiation having at least one property configured to:
i. modify at least one characteristic of at least one first cell, and
ii. at least one of (a) minimize any modification of or (b) modify at least one characteristic of at least second cell, wherein the first and second cells are different from one another, and wherein the characteristics of the first and second cells are different from one another; and
at least one second arrangement configured to determine a location associated with at least one of the at least one first cell and the at least one second cell, wherein the at least one first arrangement is further configured to forward the electromagnetic radiation in a vicinity of the location.

29. The apparatus according to claim 28, wherein at least one of the first cell or the second cell has at least one macrophage feature.

30. The apparatus according to claim 28, wherein the at least one characteristic of at least one of the at least one first cell or the at least one second cell is temperature.

31. The apparatus according to claim 28, wherein the at least one second arrangement is configured to determine the location based on an image of at least one portion of the anatomical structure.

32. The apparatus according to claim 28, wherein the at least one second arrangement includes at least one of a coherence ranging arrangement, a speckle analysis arrangement, a thermal imaging arrangement, or a spectroscopy arrangement.

33. The apparatus according to claim 28, wherein at least one of the first cell or the second cell has at least one macrophage feature, and wherein the at least one characteristic of at least one of the at least one first cell or the at least one second cell is temperature.

34. The apparatus according to claim 33, wherein the at least one property includes at least one of wavelength, average power, instanteneous power, pulse duration or total exposure duration.

35. The apparatus according to claim 34, wherein the at least one property includes a wavelength which is approximately same as a wavelength of an absoption characteristic of a compound within the at least one first cell.

36. The apparatus according to claim 34, wherein the at least one property includes a pulse duration which causes the at least one characteristic to be be confined approximately within the at least one first cell.

37. The apparatus according to claim 34, wherein the at least one property includes power which causes the at least one characteristic to irreversably damage at least one portion of the at least one first cell.

38. The apparatus according to claim 33, wherein the at least one charactristic of the at least one first cell is greater than a temperature that causes a damage to at least one portion of the at least one first cell.

39. The apparatus according to claim 38, wherein the damage to the at least one portion of the at least one first cell is irreversible.

40. The apparatus according to claim 33, wherein the at least one first cell is situated within a vascular wall.

41. The apparatus according to claim 40, wherein the vascular wall is a part of a coronary artery.

42. The apparatus according to claim 33, wherein the at least one arrangement is provided in a catheter.

45. The apparatus according to claim 33, wherein the at least one macrophage feature is a lysozome containing at least one lipid.

46. The apparatus according to claim 46, wherein the at least one lipid includes at least one low density lipo protein (“LDL”), oxidized LDL, cholesterol or cholesterol ester.

47. The apparatus according to claim 33, wherein the at least one macrophage feature is a lysozome containing nacrotic debris.

48. The apparatus according to claim 33, wherein the at least one macrophage feature is a multitude of lysozomes.

49. The apparatus according to claim 33, wherein the at least one macrophage feature is at least one nanoparticle.

50. The apparatus according to claim 33, wherein the least one nanoparticle is provided within the cell.

51. The apparatus according to claim 50, wherein the least one nanoparticle is provided within a lysozome.

52. The apparatus according to claim 50, wherein a size of a single one of the least one nanoparticle is between 1 and 20 nanometer.

53. The apparatus according to claim 50, wherein the least one nanoparticle is comprised of at least one of a metal, nobel metal, ultra-small para-magnetic iron oxide, gold or silver.

54. The apparatus according to claim 50, wherein the least one nanoparticle is administered to a subject interveneously.

55. The apparatus according to claim 50, wherein, when the electromagnetic radiation is applied on the least one nanoparticle, a surface plasmon is generated.

56. The apparatus according to claim 33, wherein the at least one first arrangement is configured to forward an electromagnetic radiation externally from a body of a subject.

57. The apparatus according to claim 33, wherein the at least one macrophage feature provided within a fiberous cap.

58. A method comprising:
forwarding an electromagnetic radiation to an anatomical structure, the electro-magnetic radiation having at least one property configured to:
iii. modify at least one characteristic of at least one first cell, and
iv. at least one of (a) minimize any modification of or (b) modify at least one characteristic of at least second cell, wherein the first and second cells are different from one another, and wherein the characteristics of the first and second cells are different from one another; and
determining a location associated with at least one of the at least one first cell and the at least one second cell, wherein the electromagnetic radiation is forwarded in a vicinity of the location.
